(19) United States
(12) Patent Application Publication
(10) Pub. No.: US 2009/0275746 A1
(43) Pub. Date: Nov. 5, 2009

(54) SOLID FAROPENEM FREE ACID

(75) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN); Dandamudi Satish Kumar, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO DRUGS LIMITED, Hyderabad, Andhrapradesh (IN)

(21) Appl. No.: 11/915,593

(22) PCT Filed: Jul. 28, 2006
(86) PCT No.: PCT/IN2006/000267

§ 371 Date: Nov. 27, 2007

(51) Int. Cl. C07D 499/861; C07D 499/04

(52) U.S. Cl. 540/310

(57) ABSTRACT

The present invention provides solid form of faropenem free acid, its hydrates and processes for their preparation thereof. Thus, for example, dissolving an alkali metal salt of faropenem in water, adjusting the pH of the solution formed with an acid to below about 2.5 and collecting the precipitated solid to obtain solid faropenem free acid.

### FIELD OF THE INVENTION

The present invention provides solid form of faropenem free acid, its hydrates and processes for their preparation thereof.

### BACKGROUND OF THE INVENTION

U.S. Pat. No. 4,997,829 disclosed penem compounds, which exhibit stronger activities against wide variety of gram-positive and gram-negative bacteria as compared with known penem compounds, processes for their production and use thereof. These compounds have very remarkable effect in treatment for infections with gram-positive and gram-negative bacteria and can be utilized broadly as medicines for human as well as animals. Among them Faropenem sodium, chemically (5R,6S)-6-[1(R)-Hydroxyethyl]-2-[2(R)-tetrahydrofuryl]penem-3-carboxylic acid monosodium salt is a orally active β-lactamase stable penem antibiotic. Faropenem sodium is a new oral and injectable penem that shows broad-spectrum antibacterial activity against both aerobic and anaerobic Gram-positive and Gram-negative bacteria, except Pseudomonas aeruginosa. Faropenem sodium is represented by the following structure:

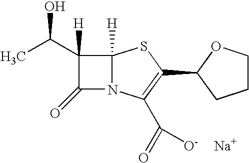

Processes for the preparations of faropenem sodium and related compounds were described in U.S. Pat. No. 4,997,829, EP Patent Application No. 0410727 A1, JP 0441489, JP 06321952, KR 9601481, Shenyang Yaoke Daxue Xuebao (2001), 18(1), 20-22, Farumashia (2002), 38(3), 219-223, and Zhongguo Yiyao Gongye Zazhi, 32(8), 339-341.

U.S. Pat. No. 4,997,829 disclosed various alkali metal salts of faropenem in solid form and processes for their preparation thereof. EP Patent Application No. 0410727 A1 disclosed alkali metal salt hydrates of faropenem, and processes for their preparation thereof. The process for the preparation of solid form of faropenem free acid is not disclosed in the prior art. We have discovered that faropenem free acid can be obtained as a solid. We have also discovered various hydrated crystalline forms of faropenem free acid. Since the crystalline faropenem free acid is obtained with high purity, the said crystalline solid can be used to obtain pharmaceutically acceptable salts of faropenem free acid in high purity. It has been found that purification of impure faropenem free acid is practically advantageous when compared with the purification of a salt of it.

One object of the present invention is to provide a solid form of faropenem free acid.

Another object of the present invention is to provide a crystalline form of faropenem free acid, process for preparing it and pharmaceutical composition comprising it.

Another object of the present invention is to provide hydrated crystalline forms of faropenem free acid, processes for preparing them and pharmaceutical compositions comprising them.

Another object of the present invention is to provide purification methods to obtain high purity faropenem free acid and pharmaceutically acceptable salts via crystalline faropenem free acid.

### DETAILED DESCRIPTION OF THE INVENTION

According to one aspect of the present invention, there is provided a solid faropenem free acid.

According to another aspect of the present invention, there is provided a process for preparation of solid faropenem free acid, which comprises isolating solid faropenem free acid from an aqueous solution of faropenem at a pH of below about 2.5.

Isolation of solid faropenem free acid may be initiated by a method usually known in the art such as cooling, seeding, partial removal of the solvent from the solution, addition of precipitating solvent or a combination thereof.

The process of the invention may be carried out by dissolving a salt of faropenem in water, adjusting the pH of the solution formed with an acid to below about 2.5 and collecting the precipitated solid to obtain solid faropenem free acid.

Preferable salt of faropenem used in the above process is an alkali metal salt and more preferable alkali metal salt is sodium or potassium salt.

The process of the invention may also be carried out by adjusting the pH of the aqueous solution of faropenem obtained as a part of the reaction mass by a known process to below 2.5 with an acid, and collecting the precipitated solid faropenem free acid.

Preferably the pH of the solution is adjusted to 1.0-2.5 and more preferably to 1.5-2.5.

Preferable acid used to adjust the pH in the above process is a mineral acid such as sulfuric acid, hydrochloric acid and phosphoric acid. More preferable mineral acid is hydrochloric acid.

Preferably aqueous solution of acid may be used to adjust the pH and more preferably dilute aqueous acid may be used.

The precipitated solid faropenem free acid is collected by filtration or centrifugation.

According to another aspect of the present invention, there is provided a crystalline solid of faropenem free acid having water content of above about 15% by weight.

According to another aspect of the present invention, there is provided a crystalline solid of faropenem free acid having water content in the range of 15-50% by weight, characterized by peaks in the powder X-ray diffraction pattern having 2θ angle positions at about 11.2, 15.1, 17.2, 18.1, 19.9, 20.8, 21.1, 22.4, 23.9, 24.2, 27.2, 29.2, 30.0 and 33.9±0.1 degrees. The typical x-ray powder diffraction pattern is shown in FIGS. 1 and 2.

According to another aspect of the present invention, a process is provided for preparation of crystalline solid of faropenem free acid having water content of above about 15% by weight, which comprises:
a) providing an aqueous solution of a faropenem salt;
b) adjusting the pH of the above solution to below about 2.5 with an acid under rapid stirring i.e., high runs per minute (RPM); and
c) collecting the crystalline faropenem free acid having water content of about above 15% by weight.

Preferably the water content of crystalline faropenem free acid obtained by the process as described above is between about 15% and 50% by weight, more preferably the water content of crystalline faropenem free acid is between about 17% and 47% by weight and still more preferably the water content of crystalline faropenem free acid is between about 17% and 20% by weight.

The crystalline faropenem free acid obtained by the process as described above has water content in the range of 15-50% by weight, and crystalline faropenem free acid shows the same characteristic powder X-ray diffraction pattern throughout this water content range.

Preferably the salt of faropenem used in step (a) is an alkali metal salt and more preferable alkali metal salt is sodium or potassium salt.

The aqueous solution of the faropenem salt may be prepared by dissolving a salt of faropenem in water or by obtaining as a part of reaction mass by a known process.

Preferably the pH of the solution obtained in step (b) is adjusted to 1.0-2.5 and more preferably to 1.5-2.5.

Preferable acid used in step (b) is a mineral acid such as sulfuric acid, hydrochloric acid and phosphoric acid. More preferable mineral acid is hydrochloric acid.

Preferably aqueous solution of acid may be used to adjust the pH and more preferably dilute aqueous acid may be used.

As used herein, the terms “rapid stirring” and “high runs per minute” are synonymous and refer to above 150 runs per minute.

The addition of acid to the solution in step (b) is preferably carried out under stirring at above about 300 runs per minute, more preferably at above about 500 runs per minute and still more preferably at above about 700 runs per minute.

The crystalline faropenem free acid obtained in step (c) is collected by filtration or centrifugation.

According to another aspect of the present invention, there is provided a crystalline solid of faropenem free acid having water content of about 8-12% by weight, characterized by peaks in the powder X-ray diffraction pattern having 2θ angle positions at about 11.1, 11.9, 13.3, 15.0, 16.6, 18.1, 19.9, 21.0, 22.3, 23.8, 24.6, 25.7, 29.1 and 33.8±0.1 degrees. The typical x-ray powder diffraction pattern is shown in FIG. 3.

According to another aspect of the present invention, a process is provided for preparation of crystalline solid of faropenem free acid having water content of about 8-12% by weight, which comprises:
a) providing an aqueous solution of a faropenem salt;
b) adjusting the pH of the above solution to below about 2.5 with an acid under slow stirring; and
c) collecting the crystalline faropenem free acid having water content of about 8-12% by weight.

The water content of crystalline faropenem free acid obtained by the process as described above is preferably between 8% and 11% by weight and more preferably between 8.5% and 10.5% by weight.

Preferably the salt of faropenem used in step (a) is an alkali metal salt and more preferable alkali metal salt is sodium or potassium salt.

The aqueous solution of the faropenem salt may be prepared by dissolving a salt of faropenem in water or by obtaining as a part of reaction mass by a known process.

Preferably the pH of the solution obtained in step (b) is adjusted to 1.0-2.5 and more preferably to 1.5-2.5.

Preferable acid used in step (b) is a mineral acid such as sulfuric acid, hydrochloric acid and phosphoric acid. More preferable mineral acid is hydrochloric acid.

Preferably aqueous solution of acid may be used to adjust the pH and more preferably dilute aqueous acid may be used.

As used herein, the term “slow stirring” refers to below 150 runs per minute.

The addition of acid to the solution in step (b) is preferably carried out under stirring at below about 140 runs per minute, more preferably at below about 130 runs per minute and still more preferably between about 100-130 runs per minute.

The crystalline faropenem free acid obtained in step (c) is collected by filtration or centrifugation.

According to another aspect of the present invention, there is provided a crystalline solid of faropenem free acid having water content of about 8-12% by weight, characterized by peaks in the powder X-ray diffraction pattern having 2θ angle positions at about 11.5, 15.4, 18.5, 20.3, 21.2, 21.4, 22.7, 24.2, 26.0, 27.2, 27.5, 30.2 and 34.2±0.1 degrees. The typical x-ray powder diffraction pattern is shown in FIG. 4.

According to another aspect of the present invention, there is provided a process for preparation of crystalline solid of faropenem free acid having water content of about 8-12% by weight, which comprises drying an faropenem free acid having water content of above about 12% at below 40° C. for a time sufficient to obtain faropenem free acid having water content of about 8-12% by weight.

The process of the invention may preferably be carried out by drying the faropenem free acid having the water content between 15% and 50% under vacuum at about 25-35° C., more preferably by drying the faropenem free acid having the water content between about 16% and 46% under vacuum at about 25-35° C. and still more preferably by drying the faropenem free acid having the water content between about 17% and 20% under vacuum at about 25-35° C.

According to another aspect of the present invention, there is provided a crystalline solid of faropenem free acid having water content of about 1.5-2.5% by weight, characterized by peaks in the powder X-ray diffraction pattern having 2θ angle positions at about 12.1, 13.4, 16.7 and 21.1±0.1 degrees. The typical x-ray powder diffraction pattern is shown in FIG. 5.

According to another aspect of the present invention, there is provided a process for preparation of crystalline solid of faropenem free acid having water content of about 1.5-2.5% by weight, which comprises drying an faropenem free acid having water content of above about 3% at below 40° C. for a time sufficient to obtain faropenem free acid having water content of about 1.5-2.5% by weight.

The process of the invention may preferably be carried out by drying the faropenem free acid having the water content between about 3% and 50% under vacuum at about 25-35° C., more preferably by drying the faropenem free acid having the water content between about 3% and 46% under vacuum at about 25-35° C. and still more preferably by drying the faropenem free acid having the water content between about 8% and 46% under vacuum at about 25-35° C.

Faropenem or its salts used as starting materials may be obtained by processes described in the art, for example by the processes described in U.S. Pat. No. 4,997,829, EP Patent Application No. 0410727 A1, JP 0441489, JP 06321952, KR 9601481, Shenyang Yaoke Daxue Xuebao (2001), 18(1), 20-22, Farumashia (2002), 38(3), 219-223, and Zhongguo Yiyao Gongye Zazhi, 32(8), 339-341.

Isolation of faropenem free acid as crystalline solid affords pure faropenem free acid, which can be converted into pharmaceutically acceptable salts of faropenem. The isolation avoids multiple purification steps of the pharmaceutically acceptable salts of faropenem.

### BRIEF DESCRIPTION OF THE DRAWINGS

FIG. 1 is an x-ray powder diffraction spectrum of crystalline solid of faropenem free acid having water content of 18% by weight obtained as per the process described in example 1.

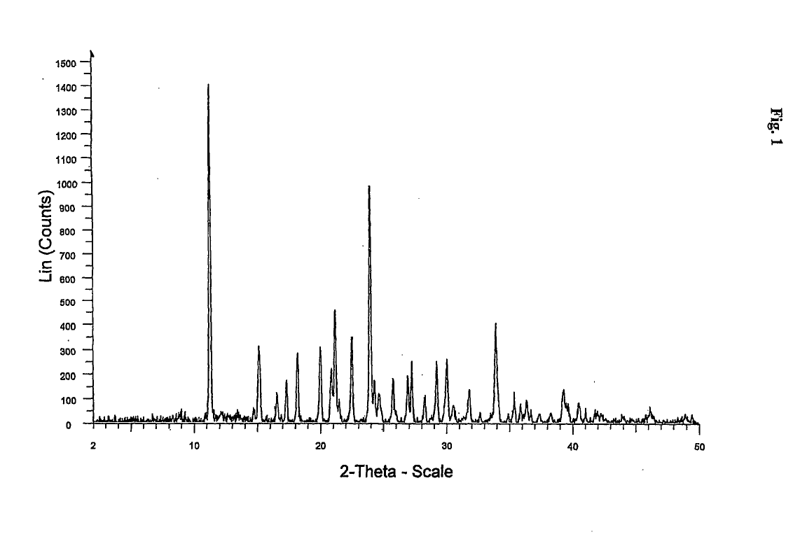

FIG. 2 is an x-ray powder diffraction spectrum of crystalline solid of faropenem free acid having water content of 45.1% by weight.

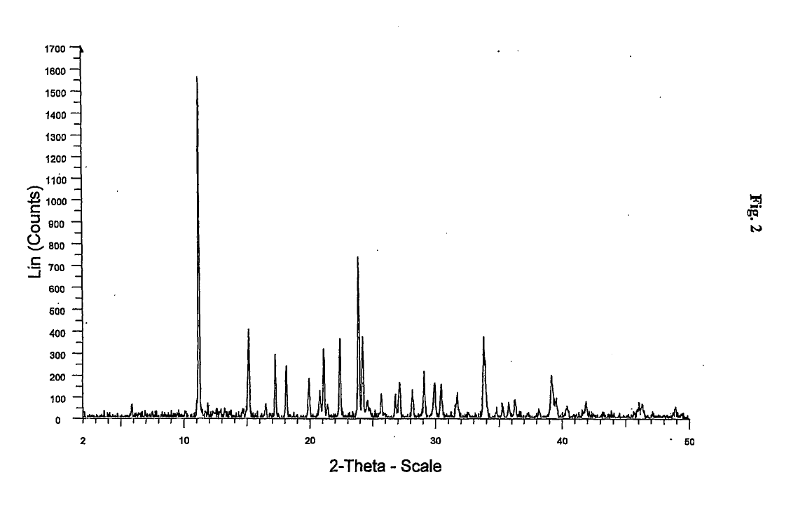

FIG. 3 is an x-ray powder diffraction spectrum of crystalline solid of faropenem free acid having water content of 9.3% by weight obtained as per the process described in example 2.

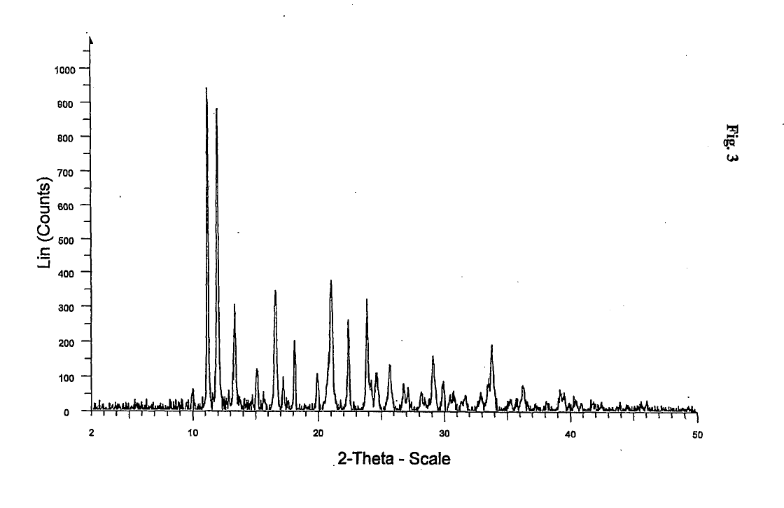

FIG. 4 is an x-ray powder diffraction spectrum of crystalline solid of faropenem free acid having water content of 9.4% by weight obtained as per the process described in example 3.

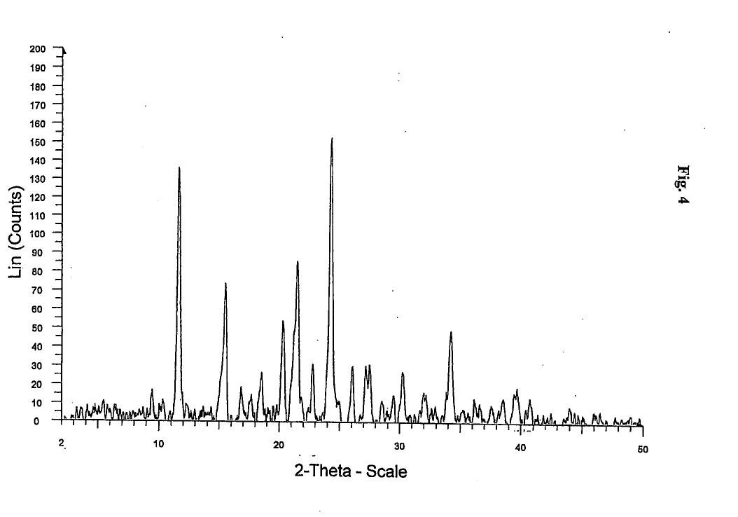

FIG. 5 is an x-ray powder diffraction spectrum of crystalline solid of faropenem free acid having water content of 2.4% by weight obtained as per the process described in example 4.

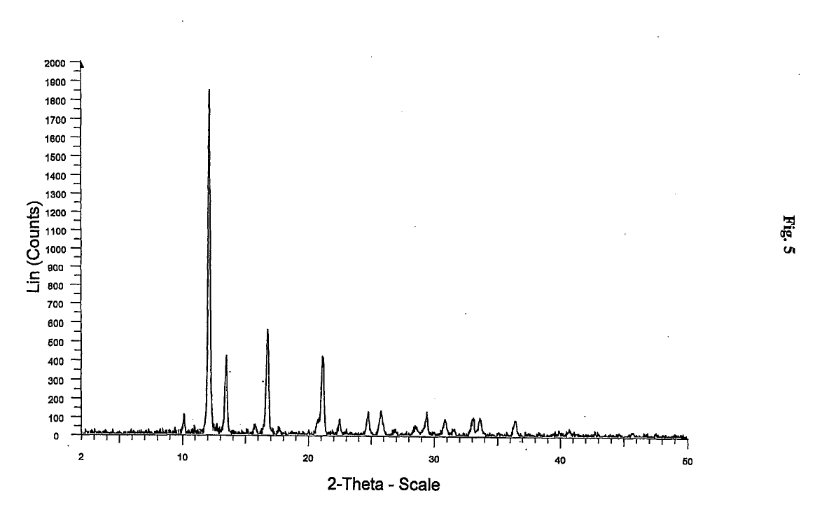

x-Ray powder diffraction spectrum was measured on a Bruker axs D8 advance x-ray powder diffractometer having a Copper-Kα radiation. Approximately 1 g of sample was gently flattened on a sample holder and scanned from 2 to 50 degrees two-theta, at 0.03 degrees two-theta per step and a step time of 0.5 seconds. The sample was simply placed on the sample holder. The sample was rotated at 30 rpm at a voltage 40 KV and current 35 mA.

The invention will now be further described by the following examples, which are illustrative rather than limiting.

### Example 1

(5R,6S)-6-[1(R)-Hydroxyethyl]-2-[2(R)-tetrahydrofuryl]penem-3-carboxylic acid 4-nitrobenzyl ester (30 gm) is dissolved in ethyl acetate (600 ml) at 25-30° C. and then 4% aqueous sodium bicarbonate solution (600 ml) is added at 25-30° C. The resulting solution is charged into autoclave, 5% Pd/C (6 gm) is added and then cooled to 20-22° C. The reaction mass is then subjected to hydrogenation while maintaining the pressure in between 6-7 Kg/cm² at 20-25° C. After completion of the hydrogenation reaction the reaction mass is filtered through hyflo bed, washed the bed with ethyl acetate (100 ml) followed by water (100 ml), the aqueous layer is separated from the filtrate and then washed two times with ethyl acetate (each time 100 ml). The aqueous layer is then treated with activated carbon (1.5 gm) at 25-30° C., filtered the mass on hyflo and washed the bed with water (100 ml). The combined filtrate is cooled to 0-5° C., bubbled with N₂ gas for 15 minutes to remove the traces of tetrahydrofuran, pH of the mass is adjusted to 1.8-2.0 with 17% aqueous hydrochloric acid and then seeded the reaction mass with pure seed of faropenem acid. The resulting mass is stirred for 1 hour, filtered the separated solid, washed the material with chilled water and then dried at 25-30° C. till the moisture content reaches to 18% to give 30 gm of pure crystalline faropenem free acid (HPLC Purity: 99.5%).

### Example 2

(5R,6S)-6-[1(R)-Hydroxyethyl]-2-[2(R)-tetrahydrofuryl]penem-3-carboxylic acid 4-nitrobenzyl ester (30 gm) is dissolved in tetrahydrofuran (600 ml) at 25-30° C. and then phosphate buffer (600 ml, pH=7) is added at 25-30° C. The resulting solution is charged into autoclave under nitrogen atmosphere, 5% Pd/C (6 gm) is added and then cooled to 20-22° C. The reaction mass is then subjected to hydrogenation while maintaining the pressure in between 6-7 Kg/cm² at 20-25° C. After completion of the hydrogenation reaction the reaction mass is filtered through hyflo bed, washed the bed with phosphate buffer (100 ml) and then pH of the filtrate is readjusted to 6.8-7.0 with 0.1 M Na₂HPO₄ solution. Distilled off tetrahydrofuran from the reaction mass under reduced pressure at 25-30° C. until no more solvent distilled out. The aqueous layer is then treated with activated carbon (1.5 gm) at 25-30° C., filtered the mass on hyflo and washed the bed with water (100 ml). The combined filtrate is cooled to 0-5° C., bubbled with N₂ gas for 15 minutes to remove the traces of tetrahydrofuran, pH of the mass is adjusted to 1.8-2.0 with 17% aqueous hydrochloric acid under very slow stirring and then seeded the reaction mass with pure seed of faropenem acid. The resulting mass is stirred for 1 hour, filtered the separated solid, washed the material with chilled water and then dried at 25-30° C. till the moisture content reaches to 9.3% w/w to give 22.5 gm of pure crystalline faropenem free acid (HPLC Purity: 99.6%).

### Example 3

Faropenem free acid (5 gm, Moisture content: 18%) is dried under vacuum at 5-10 mm/Hg for 5-6 hours at 25-30° C. to obtain faropenem free acid having the moisture content of 9.4% w/w.

### Example 4

Faropenem free acid (5 gm, Moisture content: 18%) is dried under vacuum at 5-10 mm/Hg for 24-30 hours at 25-30° C. to obtain faropenem free acid having the moisture content of 2.4% w/w.

1: Solid faropenem free acid.

2: A process for the preparation of the solid faropenem free acid of claim 1, which comprises isolating the solid faropenem free acid from an aqueous solution of faropenem at a pH of below about 2.5.

3: The process as claimed in claim 2, wherein the isolation of the solid faropenem free acid is initiated by a method such as cooling, seeding, partial removal of the solvent from the solution, addition of precipitating solvent or a combination thereof.

4: The process as claimed in claim 2, wherein the process of the invention is carried out by dissolving a salt of faropenem in water, adjusting the pH of the solution formed with an acid to below about 2.5 and collecting the precipitated solid to obtain solid faropenem free acid.

5: The process as claimed in claim 4, wherein the salt of faropenem used is an alkali metal salt.

6: The process as claimed in claim 5, wherein the alkali metal salt is sodium or potassium salt.

7: The process as claimed in claim 2, wherein the pH of the solution is adjusted to about 1.0-2.5.

8: The process as claimed in claim 7, wherein the pH of the solution is adjusted to about 1.5-2.5.

9: The process as claimed in claim 2, wherein the acid used to adjust the pH is a mineral acid such as sulfuric acid, hydrochloric acid and phosphoric acid.

10: The process as claimed in claim 9, wherein the mineral acid is hydrochloric acid.

11: The process as claimed in claim 2, wherein an aqueous solution of acid is used to adjust the pH.

12: The process as claimed in claim 11, wherein a dilute aqueous solution of acid is used to adjust the pH.

13: The process as claimed in claim 2, wherein the precipitated solid faropenem free acid is collected by filtration or centrifugation.

14: Crystalline solid of faropenem free acid.

15: Crystalline solid of faropenem free acid having water content of above about 15% by weight.

16: The compound as claimed in claim 15, wherein the crystalline solid of faropenem free acid having a water content in the range of about 15-50% by weight, is characterized by peaks in the powder X-ray diffraction pattern having 2θ angle positions at about 11.2, 15.1, 17.2, 18.1, 19.9, 20.8, 21.1, 22.4, 23.9, 24.2, 27.2, 29.2, 30.0 and 33.9±0.1 degrees.

17: A process for the preparation of the crystalline solid of faropenem free acid having a water content of above about 15% by weight of claim 15, which comprises:
a) providing an aqueous solution of a faropenem salt;
b) adjusting the pH of the solution to below about 2.5 with an acid under rapid stirring; and
c) collecting the crystalline faropenem free acid having water content of about above 15% by weight.

18: The process as claimed in claim 17, wherein the water content of crystalline faropenem free acid obtained is between about 15% and 50% by weight.

19: The process as claimed in claim 18, wherein the water content of crystalline faropenem free acid is between about 17% and 47% by weight.

20: The process as claimed in claim 19, wherein the water content of crystalline faropenem free acid is between about 17% and 20% by weight.

21: The process as claimed in claim 17, wherein the salt of faropenem used in step (a) is an alkali metal salt such as a sodium or potassium salt.

22: The process as claimed in claim 17, wherein the aqueous solution of the faropenem salt is prepared by dissolving a salt of faropenem in water.

23: The process as claimed in claim 17, wherein the pH of the solution obtained in step (b) is adjusted to about 1.0-2.5.

24: The process as claimed in claim 23, wherein the pH of the solution is adjusted to about 1.5-2.5.

25: The process as claimed in claim 17, wherein the acid used in step (b) is a mineral acid such as sulfuric acid, hydrochloric acid and phosphoric acid.

26: The process as claimed in claim 25, wherein the mineral acid is hydrochloric acid.

27: The process as claimed in claim 17, wherein the aqueous solution of acid is used to adjust the pH.

28: The process as claimed in claim 27, wherein the dilute aqueous solution of acid is used to adjust the pH.

29: The process as claimed in claim 17, wherein the addition of acid to the solution in step (b) is carried out under stirring at above about 300 runs per minute.

30: The process as claimed in claim 29, wherein the addition of acid to the solution in step (b) is carried out under stirring at above about 500 runs per minute.

31: The process as claimed in claim 30, wherein the addition of acid to the solution in step (b) is carried out under stirring at above about 700 runs per minute.

32: The process as claimed in claim 17, wherein the crystalline faropenem free acid obtained in step (c) is collected by filtration or centrifugation.

33: Crystalline solid of faropenem free acid having a water content of about 8-12% by weight, characterized by peaks in the powder X-ray diffraction pattern having 2θ angle positions at about 11.1, 11.9, 13.3, 15.0, 16.6, 18.1, 19.9, 21.0, 22.3, 23.8, 24.6, 25.7, 29.1 and 33.8±0.1 degrees.

34: A process for the preparation of the crystalline solid of faropenem free acid having the water content of about 8-12% by weight of claim 33, which comprises:
a) providing an aqueous solution of a faropenem salt;
b) adjusting the pH of the above solution to below about 2.5 with an acid under slow stirring; and
c) collecting the crystalline faropenem free acid having a water content of about 8-12% by weight.

35: The process as claimed in claim 34, wherein the water content of crystalline faropenem free acid obtained is between about 8% and 11% by weight.

36: The process as claimed in claim 35, wherein the water content of crystalline faropenem free acid is between about 8.5% and 10.5% by weight.

37: The process as claimed in claim 34, wherein the salt of faropenem used in step (a) is an alkali metal salt such as sodium or potassium salt.

38: The process as claimed in claim 34, wherein the aqueous solution of the faropenem salt is prepared by dissolving a salt of faropenem in water.

39: The process as claimed in claim 34, wherein the pH of the solution obtained in step (b) is adjusted to about 1.0-2.5.

40: The process as claimed in claim 39, wherein the pH of the solution is adjusted to about 1.5-2.5.

41: The process as claimed in claim 34, wherein the acid used in step (b) is a mineral acid such as sulfuric acid, hydrochloric acid and phosphoric acid.

42: The process as claimed in claim 41, wherein the mineral acid is hydrochloric acid.

43: The process as claimed in claim 34, wherein the aqueous solution of acid is used to adjust the pH.

44: The process as claimed in claim 34, wherein the addition of acid to the solution in step (b) is carried out under stirring at below about 140 runs per minute.

45: The process as claimed in claim 44, wherein the addition of acid to the solution in step (b) is carried out under stirring at below about 130 runs per minute.

46: The process as claimed in claim 45, wherein the addition of acid to the solution in step (b) is carried out under stirring at between about 100-130 runs per minute.

47: The process as claimed in claim 34, wherein the crystalline faropenem free acid obtained in step (c) is collected by filtration or centrifugation.

48: Crystalline solid of faropenem free acid having a water content of about 8-12% by weight, characterized by peaks in the powder X-ray diffraction pattern having 2θ angle positions at about 11.5, 15.4, 18.5, 20.3, 21.2, 21.4, 22.7, 24.2, 26.0, 27.2, 27.5, 30.2 and 34.2±0.1 degrees.

49: A process for the preparation of the crystalline solid of faropenem free acid having the water content of about 8-12% by weight of claim 48, which comprises drying an faropenem free acid having a water content of above about 12% at below 40° C. for a time sufficient to obtain faropenem free acid having water content of about 8-12% by weight.

50: The process as claimed in claim 49, wherein the process is carried out by drying the faropenem free acid having the water content between about 15% and 50% under vacuum at about 25-35° C.

51: The process as claimed in claim 50, wherein the process is carried out by drying the faropenem free acid having a water content between about 16% and 46% under vacuum at about 25-35° C.

52: The process as claimed in claim 51, wherein the process is carried out by drying the faropenem free acid having a water content between about 17% and 20% under vacuum at about 25-35° C.

53: Crystalline solid of faropenem free acid having a water content of about 1.5-2.5% by weight, characterized by peaks in the powder X-ray diffraction pattern having 2θ angle positions at about 12.1, 13.4, 16.7 and 21.1±0.1 degrees.

54: A process for the preparation of the crystalline solid of faropenem free acid having water content of about 1.5-2.5% by weight of claim 53, which comprises drying an faropenem free acid having a water content of above about 3% at below 40° C. for a time sufficient to obtain faropenem free acid having a water content of about 1.5-2.5% by weight.

55: The process as claimed in claim 54, wherein the process is carried out by drying the faropenem free acid having a water content between 3% and 50% under vacuum at about 25-35° C.

56: The process as claimed in claim 55, wherein the process is carried out by drying the faropenem free acid having a water content between 3% and 46% under vacuum at about 25-35° C.

57: The process as claimed in claim 56, wherein the process is carried out by drying the faropenem free acid having a water content between about 8% and 46% under vacuum at about 25-35°* C.
